SEQUENCE LISTING (13 sequences)

SEQ ID NO: 1 - protein, 5 aa, Artificial Sequence
  Synthetic Peptide Fragment
FYWDP

SEQ ID NO: 2 - protein, 5 aa, Artificial Sequence
  Synthetic Peptide Fragment
FLVFK

SEQ ID NO: 3 - protein, 5 aa, Artificial Sequence
  Synthetic Peptide Fragment
WLWKW

SEQ ID NO: 4 - protein, 5 aa, Artificial Sequence
  Synthetic Peptide Fragment
FYWDP

SEQ ID NO: 5 - protein, 6 aa, Artificial Sequence
  Synthetic Peptide Fragment
WKFRYK

SEQ ID NO: 6 - protein, 6 aa, Artificial Sequence
  Synthetic Peptide Fragment
LYFRRW

SEQ ID NO: 7 - protein, 6 aa, Artificial Sequence
  Synthetic Peptide Fragment
LRLKYR

SEQ ID NO: 8 - protein, 6 aa, Artificial Sequence
  Synthetic Peptide Fragment
LYRRFY

SEQ ID NO: 9 - protein, 6 aa, Artificial Sequence
  Synthetic Peptide Fragment
KFRFRY

SEQ ID NO: 10 - protein, 6 aa, Artificial Sequence
  Synthetic Peptide Fragment
GRFRLK

SEQ ID NO: 11 - protein, 6 aa, Artificial Sequence
  Synthetic Peptide Fragment
YRRWFR

SEQ ID NO: 12 - protein, 6 aa, Artificial Sequence
  Synthetic Peptide Fragment
LYFYKR

SEQ ID NO: 13 - protein, 6 aa, Artificial Sequence
  Synthetic Peptide Fragment
GRFRLF

(19) United States
(12) Patent Application Publication
(10) Pub. No.: US 2013/0267423 A1
(43) Pub. Date: Oct. 10, 2013

(54) PEPTIDE LIBRARIES FOR SCREENING AND OTHER APPLICATIONS

(71) Applicants: Su Seong Lee, Singapora (SG); Jaehong Lim, Singapore (SG); Junhoe Cha, Singapore (SG); Yi Li Ang, Singapore (SG); Yiran Zheng, Singapore (SG); Sylvia Tan, Singapore (SG)

(72) Inventors: Su Seong Lee, Singapora (SG); Jaehong Lim, Singapore (SG); Junhoe Cha, Singapore (SG); Yi Li Ang, Singapore (SG); Yiran Zheng, Singapore (SG); Sylvia Tan, Singapore (SG)

(21) Appl. No.: 13/819,534

(22) PCT Filed: Aug. 29, 2011
(86) PCT No.: PCT/SG11/00298

§ 371 Date: Jun. 11, 2013

(30) Foreign Application Priority Data
Aug. 27, 2010 (SG) 201006269-3

(51) Int. Cl. G01N 33/68

(52) U.S. Cl. CPC G01N 33/6818
USPC 506/2; 506/18; 506/12

(57) ABSTRACT

The present invention generally relates to various peptides and particles, for example, for use in screening of particle- or bead-based peptide libraries. In one aspect, the present invention is generally directed to articles including peptides attached to one or more particles, which may have structures such as (particle)-M-Q-Zₙ-X-J, (particle)-M-Q-Zₙ—X¹—X²—X³—X⁴—X⁵-J, (particle)-M-R—Zₙ—X¹—X²—X³—X⁴—X⁵-J, (particle)-M-R—X¹—X²—X³—X⁴—X⁵Zₙ-Q-J, (particle)-M-X¹—X²—X—³X⁴—X⁵—Zₙ—R-J, etc., where M is a methionine residue, M1 is a cleavable linker residue, Q is a group able to enhance intensity and/or sensitivity of mass spectrometry, X comprises one or more amino acid residues, n is a positive integer, Z is a covalent bond or a spacer, and J is an end-group. In some embodiments, the spacer may comprise a structure such as:(I). Other aspects of the present invention generally relate to methods of using such articles, e.g., by exposing the article to a target molecule such as a protein, for example, for use in screening of particle-based peptide libraries. Still other aspects of the present invention generally relate to methods of making such articles, methods of promoting such articles, kits involving such articles, or the like.

### RELATED APPLICATIONS

This application claims the benefit of Application No. 201006269-3, filed Aug. 27, 2010, entitled “Peptide Libraries for Screening and Other Applications,” by Lee, et al., incorporated herein by reference.

### FIELD OF INVENTION

The present invention generally relates to various peptides and particles, for example, for use in screening of particle-based peptide libraries.

### BACKGROUND

Screening of OBOC (one-bead-one-compound) combinatorial libraries is a powerful and effective method for the rapid identification of synthetic ligands against molecular or cellular targets. In pursuit of high throughput screening of OBOC peptide libraries with target molecules, a few existing problems need to be addressed.

On-bead screening of the OBOC library is often vulnerable to interference from non-specific bindings between the ligand and the target object, which often results in biased screening information and false hit sequences. Most protein targets tend to bind to the peptides containing a number of positively charged amino acids. Among those, such as arginine (R), lysine (K) and histidine (H), the occurrence of R is the most rampant. For example, prior studies on profiling the sequences specific to Src homology 2 (SH2) and PDZ domains have found that protein domains tend to pick out many hit sequences that are rich in positively charged amino acids. Upon testing, however, it was shown that the positively charged amino acids residues contribute minimally, if not none at all, to overall binding. This phenomenon was largely attributed to non-specific interactions occurring between the target and the ligand.

De novo sequencing of peptides and proteins is a well-established method with high accuracy. In the case of sequencing peptides, the main strategy employed is to obtain the mass spectra of a series of peptide fragment ions of a peptide with a known parent mass. Sequencing is then done based on the difference in mass values of fragmented peptides from MS/MS spectra. Hence, the efficiency and accuracy depend significantly on the quality of the MS/MS spectra. There are also a few other common problems associated with ionization efficiency and range of parent mass. The peptide signal intensities of MS and MS/MS in MALDI-TOF/TOF experiments are often varying and unpredictable. The signal intensity for MALDI-generated ions is a function of several intrinsic properties of peptide sequences. These properties include charge of the residue (e.g., guanidine in arginine), length, hydrophobicity, and secondary structure of a particular peptide.

One of the most serious problems associated with MALDI-TOF/TOF-based sequencing of OBOC library is that the peptide fragments containing acidic residues tend to have low signal intensities. The negatively charged groups reduce ionization efficiency and in turn cause low signal-to-noise ratios. There are existing methods to improve the peak intensities of peptide fragments containing such groups, for example, enhancing MALDI-TOF MS signals of a peptide 20- to 35-fold upon picolinamidination. Picolinamidination of amino groups in peptides uses ethyl picolinimidate tetrafluoroborate which is synthesized from picolinamide and triethyloxonium tetrafluoroborate. The N-terminus of the peptide chain and the epsilon-amino group of lysine will undergo the abovementioned reaction. Other approaches include the addition of coumarin tags on the N-terminus of peptide chain, which could potentially enhance the signal intensity by up to 40-fold. These enhancement methods involve the modification of the N-terminus of the peptide chains. This is usually undesirable for ligand- or capture agent-related applications using OBOC library as structural modifications might change the ligand-substrate binding modes.

Accordingly, improvements to screening of OBOC (one-bead-one-compound) combinatorial libraries are still needed.

### SUMMARY

The present invention generally relates to various peptides and particles, for example, for use in screening of particle-based peptide libraries. The subject matter of the present invention involves, in some cases, interrelated products, alternative solutions to a particular problem, and/or a plurality of different uses of one or more systems and/or articles.

In one aspect, the present invention is directed to an article. In accordance with one set of embodiments, the article comprises a particle having a peptide attached thereto, having a structure:

(particle)-M-Q-Zₙ—X-J,

where M is a methionine residue; Q is a group able to enhance intensity and/or sensitivity of mass spectrometry; X comprises a plurality of amino acid residues; n is a positive integer; J is an endgroup; and Z is a spacer. In some embodiments, Z has a structure:

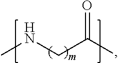

where m is a positive integer greater than or equal to 3.

In another set of embodiments, the article comprises a collection of particles having peptides attached thereto. At least some of the particles may consist essentially of one type of peptide. The collection of particles may include at least 20 distinguishable types of peptides, where the particles and the peptides have a structure:

(particle)-M-Q-Zₙ—X¹—X²—X³—X⁴—X⁵-J,

where M is a methionine residue; Q is a group able to enhance intensity and/or sensitivity of mass spectrometry; Zₙ is either a covalent bond or a spacer that does not contain a naturally-occurring amino acid residue, where n is a positive integer greater than or equal to 2 when Z is a spacer; X¹, X², X³, X⁴, and X⁵ are each independently amino acid residues excluding arginine; and J is an endgroup.

The article, in yet another set of embodiments, includes a particle having a peptide attached thereto, having a structure:

(particle)-M-R—Z₂—X¹—X²—X³—X⁴—X⁵-J,

where M is a methionine residue; R is an arginine residue; X¹, X², X³, X⁴, and X⁵ are each independently amino acid residues excluding arginine; J is an endgroup; and Z is a spacer. In one set of embodiments, Z may have a structure:

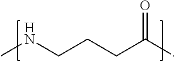

The article, in still another set of embodiments, includes a particle having a peptide attached thereto, having a structure:

(particle)-M-X¹—X²—X³—X⁴—X⁵-Zₙ-Q-J,

where M is a methionine residue; X¹, X², X³, X⁴, and X⁵ are each independently amino acid residues excluding arginine and cysteine; Zₙ is either a covalent bond or a spacer that does not contain a naturally-occurring amino acid residue, where n is a positive integer when Z is a spacer; Q is either a covalent bond between Zₙ and J, or a group able to enhance intensity and/or sensitivity of mass spectrometry; and J is an endgroup.

In another set of embodiments, the article comprises a particle having a peptide attached thereto, having a structure:

(particle)-M¹-X¹—X²—X³—X⁴—X⁵-Zₙ—R-J,

where M¹ is a cleavable linker residue; X¹, X², X³, X⁴, and X⁵ are each independently amino acid residues excluding arginine and cysteine; Zₙ is either a covalent bond or a spacer that does not contain a naturally-occurring amino acid residue, wherein n is a positive integer when Z is a spacer; Q is either a covalent bond between Zₙ and J, or a group able to enhance intensity and/or sensitivity of mass spectrometry; and J is an endgroup.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a particle (represented by a ball) comprising a peptide sequence, in one embodiment of the invention;

FIGS. 2A-2B illustrate mass spectrometry data for a peptide comprising FLVFK (SEQ ID NO: 2), in accordance with one embodiment of the invention;

FIGS. 3A-3B illustrate mass spectrometry data for a peptide comprising WLWKW (SEQ ID NO: 3), in accordance with another embodiment of the invention;

FIGS. 4A-4B illustrate mass spectrometry data for a peptide comprising FYWDP (SEQ ID NO: 4), in accordance with yet another embodiment of the invention;

FIGS. 5A-5B illustrate mass spectrometry data for a peptide comprising FYWDP (SEQ ID NO: 1), in accordance with still another embodiment of the invention;

FIGS. 6A-6B illustrate various font histograms for certain amino acids in accordance with various embodiments of the invention;

FIGS. 7A-7B illustrate certain particles comprising peptide sequence, in yet another embodiment of the invention;

FIGS. 8A-8B illustrate SPR and dot blots, respectively, of peptides in accordance with certain embodiments of the invention; and

FIG. 9 illustrates a reaction scheme used to synthesize certain structures in accordance with various embodiments of the invention.

### BRIEF DESCRIPTION OF THE SEQUENCES

SEQ ID NO: 1 is FYWDP, a fragment of a peptide;

SEQ ID NO: 2 is FLVFK, a fragment of a peptide;

SEQ ID NO: 3 is WLWKW, a fragment of a peptide;

SEQ ID NO: 4 is FYWDP, a fragment of a peptide;

SEQ ID NO: 5 is WKFRYK, a fragment of a peptide;

SEQ ID NO: 6 is LYFRRW, a fragment of a peptide;

SEQ ID NO: 7 is LRLKYR, a fragment of a peptide;

SEQ ID NO: 8 is LYRRFY, a fragment of a peptide;

SEQ ID NO: 9 is KFRFRY, a fragment of a peptide;

SEQ ID NO: 10 is GRFRLK, a fragment of a peptide;

SEQ ID NO: 11 is YRRWFR, a fragment of a peptide;

SEQ ID NO: 12 is LYFYKR, a fragment of a peptide; and

SEQ ID NO: 13 is GRFRLF, a fragment of a peptide.

### DETAILED DESCRIPTION

The present invention generally relates to various peptides and particles, for example, for use in screening of particle- or bead-based peptide libraries. In one aspect, the present invention is generally directed to articles including peptides attached to one or more particles, which may have structures such as (particle)-M-Q-Zₙ—X-J. (particle)-M-Q-Zₙ—X¹—X²—X³—X⁴—X⁵-J, (particle)-M-R-Z₂-X¹—X²—X³—X⁴—X⁵-J, (particle)-M-X¹—X²—X³—X⁴—X⁵—Zₙ-Q-J, (particle)-M¹-X¹—X²—X³—X⁴—X⁵—Zₙ—R-J. etc., where M is a methionine residue, M¹ is a cleavable linker residue, Q is a group able to enhance intensity and/or sensitivity of mass spectrometry, X comprises one or more amino acid residues, n is a positive integer, Z is a covalent bond or a spacer, and J is an endgroup. In some embodiments, the spacer may comprise a structure such as:

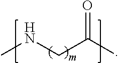

Other aspects of the present invention generally relate to methods of using such articles, e.g., by exposing the article to a target molecule such as a protein, for example, for use in screening of particle-based peptide libraries. Still other aspects of the present invention generally relate to methods of making such articles, methods of promoting such articles, kits involving such articles, or the like.

In one aspect, the present invention is generally directed to particles comprising one or more peptides attached thereto. It should be understood that, as used herein, a “peptide” is not to be limited to only sequences formed from only naturally-occurring amino acids, and the peptide may also include unnatural amino acids, or other elements that can be incorporated within a peptide, e.g., elements comprising an —NH₂ moiety and a —COOH moiety that can be incorporated via peptide bonds into the peptide. e.g., in a linear fashion. In some cases, by using such peptides, MS (mass spectrometry) signals obtained when analyzing such particles, for example, in screening of particle-based peptide libraries, may be enhanced. In one set of embodiments, the peptide attached to the protein may include a cleavable linker residue (such as methionine), a group able to enhance intensity and/or sensitivity of mass spectrometry (for example, arginine or an —NH₂ moiety), and a plurality of amino acid residues. In some cases, the peptide may also include one, two, or more spacers. In some embodiments, the spacers do not contain a naturally-occurring amino acid residue. In one set of embodiments, a spacer may have a structure:

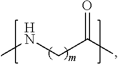

where m is a positive integer. For example, when m is 3, then the structure is gamma-amino butyric acid, or GABA.

In some embodiments, the peptide may include a cleavable linker residue, a group able to enhance intensity and/or sensitivity of mass spectrometry, an optional spacer, and a plurality of amino acid residues, in any suitable order. For example, the peptide may include (extending away from the surface of a particle) a cleavable linker residue, a group able to enhance intensity and/or sensitivity of mass spectrometry, one or more spacers, and a plurality of amino acid residues, and an endgroup; the peptide may include a cleavable linker residue, a plurality of amino acid residues, one or more spacers, a group able to enhance intensity and/or sensitivity of mass spectrometry, and an endgroup; or the peptide may include a cleavable linker residue, a group able to enhance intensity and/or sensitivity of mass spectrometry, a plurality of amino acid residues, and one or more spacers, and an endgroup.

The particle (or “bead”) may be formed of any suitable material, depending on the application. For example, the particles may comprise a glass, and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, a plurality of particles may be used, and in some cases, some, or substantially all, of the particles may be the same. For example, at least about 10%, at least about 30%, at least about 40%, at least about 50%, at least about 60%, at least about 70%, at least about 80%, at least about 90%, at least about 95%, or at least about 99% of the particles may have the same shape, and/or may have the same composition.

The particles may also have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. The particles may be spherical or non-spherical. The average diameter of a non-spherical particle is the diameter of a perfect sphere having the same volume as the non-spherical particle. In one embodiment, the particle is a spherical bead.

In some cases, there may be a collection of particles having peptides attached thereto, at least some of which are different from other particles in the collection. For example, there may be a collection of particles each consisting essentially of one type of peptide, where the peptides are different between at least some of the particles, e.g., to form a library such as a peptide library. In some cases, for example, the collection of particles may include at least 10, at least 20, at least 30, at least 50, at least 100, at least 300, at least 1,000, at least 3.000, or at least 10,000 distinguishable types of peptides. Those of ordinary skill in the art will be aware of suitable techniques for producing an array of peptides and attaching the peptides onto particles.

In certain embodiments, the peptide may be attached to a particle using a cleavable linker residue. Methionine is one example of a cleavable linker residue, although in other embodiments, other cleavable linker residues may be used. In some cases, the cleavable linker residue is cleavable upon exposure to cyanogen bromide. Other non-limiting examples of cleavable linker residues and corresponding cleaving agents that can be used to cleave the cleavable linker residues include, but are not limited to, BNPS-skatole or 2-(2′-nitrophenylsulfenyl)-3-methyl-3-bromoinolenine, which cleaves at tryptophan (Trp) residues; formic acid, which cleaves at aspartic acid-proline (Asp-Pro) peptide bonds; hydroxylamine, which cleaves at asparagine-glycine (Asn-Gly) peptide bonds; or 2-nitro-5-thiocyanobenzoic acid (NTCB), which cleaves at cysteine (Cys) residues.

In some embodiments, the peptide may include a group able to enhance intensity and/or sensitivity of mass spectrometry, e.g., for identification of the peptide during mass spectrometry analysis. For example, the group may comprise an arginine (R) residue such as described herein. As other examples, the group may be positively charged, or comprise a positively charged residue, e.g., lysine (K), or the group may be any suitable group that includes bromine therein (—Br), and/or chlorine therein (—Cl). In some cases, the group may be modifiable to give characteristic peaks in a MS/MS spectrum, such as the loss of ammonia other to have satellite peaks of −17 amu in fragments containing it, e.g., from arginine or other suitable groups. Bromine may be useful in certain embodiments due to the characteristic of bromine that comprises two similar portions of 79 and 81 amu isotopes for easy identification of fragments. Chlorine may be similarly useful because chlorine comprises two similar portions of 35 and 37 amu isotopes for easy identification of fragments. Thus, any suitable group containing bromine and/or chlorine may be used as a group able to enhance intensity and/or sensitivity of mass spectrometry, in various embodiments.

In some (but not all) embodiments, the peptide may include one or more spacers. In some cases, the spacer, when present, may be used in the peptide to increase the mass of the peptide. In some cases, a spacer is optional, and in certain formulae discussed herein, two elements connected by a spacer may also be connected, in other embodiments, by a covalent bond.

In some embodiments, the spacer may be chosen to be compatible with amino acids (e.g., comprising an —NH₂ moiety and a —COOH moiety, so that it can be incorporated via peptide bonding to the rest of the peptide), e.g., in a linear fashion. However, in certain instances, the spacer may be chosen to include unnatural amino acids and/or non-alpha amino acids, e.g., to avoid confusion with other amino acid residues present within the peptide. For example, in one set of embodiments, the spacer may comprise a structure:

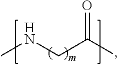

where m is a positive integer. In some cases, m is a positive integer greater than or equal to 3. For example, m may be 3, 4, 5, 6, etc. As a specific example, in one set of embodiments, m may be 3, i.e., the spacer may be gamma-amino butyric acid (“GABA”):

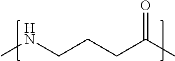

However, the use of GABA as a spacer is by way of example only, and in other embodiments, no spacer may be present, and/or a spacer may be used that does not contain a naturally-occurring amino acid residue. Other non-limiting examples of spacers include an aromatic group (e.g., having a benzene ring). For example, in one embodiment, the spacer comprises ortho, meta, or para aminobenzoic acid.

There may be 0, 1, 2, 3, 4, or any other suitable number of spacers present. If more than one spacer is present, the spacers may independently be the same or different. The spacers may be positioned next to each other in the peptide, and/or positioned in different locations in the peptide. In some cases, identical spacers are used to simplify analysis of the resulting MS measurements.

The peptide attached to the particle may also include a plurality of amino acid residues. For example, the peptide may have 1, 2, 3, 4, 5, 6, 7, 8, 9, 10, 11. 12, 13, 14, 15, 16, 17, 18, 19, and/or 20 residues. For example, the peptide may have 5 or 6 amino acid residues. However, in other embodiments, the peptide may have more or fewer numbers of residues. For instance, there may be between 4 and 10 amino acid residues present on the peptide.

The amino acid residues may be naturally occurring or non-naturally occurring. The “naturally-occurring amino acids,” as used herein, are the 20 amino acids commonly found in nature, typically in the L-isomer, i.e., alanine (“Ala” or “A”), arginine (“Arg” or “R”), asparagine (“Asn” or “N”), aspartic acid (“Asp” or “D”), cysteine (“Cys” or “C”), glutamine (“Gln” or “Q”), glutamic acid (“Glu” or “E”), glycine (“Gly” or “G”), histidine (“His” or “H”), isoleucine (“Ile” or “I”), leucine (“Leu” or “L”), lysine (“Lys” or “K”), methionine (“Met” or “M”), phenylalaine (“Phe” or “F”), proline (“Pro” or “P”), serine (“Ser” or “S”), threonine (“Thr” or “T”). tryptophan (“Trp” or “W”), tyrosine (“Tyr” or “Y”), and valine (“Val” or “V”). In some cases, an amino acid that can be either asparagine or aspartic acid is referred to as “Asx” or “B,” while an amino acid that can be either glutamine or glutamic acid is referred to as “Glx” or “Z.”

A “non-naturally-occurring amino acid,” as used herein, is an amino acid or an imino acid that is not one of the natural amino acids. Non-limiting examples of unnatural amino acids include alloisoleucine, allothreonine, homophenylalanine, homoserine, homocysteine, 5-hydroxylysine, 4-hydroxyproline, 4-carboxyglutamic acid, cysteic acid, cyclohexylalanine, ethylglycine, norleucine, norvaline, 3-aminobutyric acid, beta-amino acids (e.g., beta-alanine), N-methylated amino acids such as N-methylglycine, N-methylalanine, N-methylvaline, N-methylleucine, N-methylisoleucine, N-methylnorleucin, N-methyl-2-aminobutyric acid, N-methyl-2-aminopentanoic acid, etc., as well as the D-isomers of the natural amino acids (with the exception of glycine, which is identical to its L-isomer).

In some cases, amino acids may be selected to form peptides where arginine is excluded. In certain embodiments, arginine and cysteine may be excluded, and in some instances, arginine, cysteine, and methionine may be excluded. For example, 1, 2, 3, 4, 5, 6, or all of the plurality of amino acid residues within the peptide may exclude arginine; may exclude arginine and cysteine; or may exclude arginine, cysteine, and methionine.

The peptide may terminate with any suitable endgroup. For example, the endgroup may be an acetyl. Other endgroups on the peptide besides acetyl may be used in other embodiments, for example, —H or —OH moieties, amine or amide moieties, or the like. The endgroup is relatively unimportant in some embodiments, so long as it does not adversely interfere in MS analysis of the peptides or particles.

As specific non-limiting examples of the above described peptides, in one set of embodiments, a peptide attached to a particle may have a structure:

(particle)-M-Q-Zₙ—X-J,

where M is methionine or another cleavable linker residue. Q is a group able to enhance intensity and/or sensitivity of mass spectrometry (for example, arginine or any other such group discussed herein), X comprises a plurality of amino acid residues (e.g., 5, 6, or any other suitable number), n is a positive integer, J is an endgroup (for example acetyl or —OH, or any other such group discussed herein), and Z is a spacer. See above for a fuller discussion of each of these. For example. Z may have a structure:

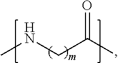

where m is a positive integer (e.g., greater than or equal to 3), or Z may have a structure as discussed herein. If n is 2 or more, then each spacer may independently be the same or different.

As another example, the structure may be:

(particle)-M-Q-Zₙ—X¹—X²—X³—X⁴—X⁵-J,

where M is methionine or another cleavable linker residue, Q is a group able to enhance intensity and/or sensitivity of mass spectrometry, Zₙ is either a covalent bond or a spacer that does not contain a naturally-occurring amino acid residue (where n is a positive integer, e.g., 1, 2, 3, or more, when Z is a spacer), X¹, X², X³, X⁴, and X⁵ are each independently amino acid residues excluding arginine, and J is an endgroup. Again, see above for a fuller discussion of each of these.

As yet another example, the structure may be:

(particle)-M-R—Z₂—X¹—X²—X³—X⁴—X⁵-J,

where M is a methionine or another cleavable linker residue, R is an arginine residue or group able to enhance intensity and/or sensitivity of mass spectrometry. X¹, X², X³, X⁴, and X⁵ are each independently amino acid residues excluding arginine, J is an endgroup, and each Z is a spacer. The spacers may be the same or different. For example, one or both Z's may be a GABA residue, or any other spacer described herein. See above for a fuller discussion of each of these.

As still another example, the structure may be:

(particle)-M-X¹—X²—X³—X⁴—X⁵—Zₙ-Q-J,

where M is a methionine or another cleavable linker residue. X¹, X², X³, X⁴, and X⁵ are each independently amino acid residues excluding arginine and cysteine, Zₙ is either a covalent bond or a spacer that does not contain a naturally-occurring amino acid residue (where n is a positive integer, e.g., 1, 2, 3, or more, when Z is a spacer). Q is either a covalent bond between Zₙ and J, or a group able to enhance intensity and/or sensitivity of mass spectrometry, and J is an endgroup. See above for a fuller discussion of each of these.

As another example, the structure may be:

(particle)-M¹-X¹—X²—X³—X⁴—X⁵—Zₙ—R-J,

where M¹ is a methionine or another cleavable linker residue, X¹, X², X³, X⁴, and X⁵ are each independently amino acid residues excluding arginine and cysteine, Zₙ is either a covalent bond or a spacer that does not contain a naturally-occurring amino acid residue (where n is a positive integer, e.g., 1, 2, 3, or more, when Z is a spacer), Q is either a covalent bond between Zₙ and J, or a group able to enhance intensity and/or sensitivity of mass spectrometry, and J is an endgroup. See above for a fuller discussion of each of these.

As previously discussed, the structures immediately above are provided by way of example only, and should not be construed as limiting. Other arrangements of elements within the peptide attached to the protein are also contemplated, as noted above.

The following documents are incorporated herein by reference: Singaporean Apl. No. 201006269-3, filed Aug. 27, 2010, entitled “Peptide Libraries for Screening and Other Applications,” by Lee, et al.; and International Patent Application No. PCT/SG2010/000266. filed Jul. 15, 2010, entitled “Method for the Improved Screening of Bead-Based Peptide and Peptide Mimetic Libraries Using Partially Cleavable Peptides,” by Heath, et al.

The following examples are intended to illustrate certain embodiments of the present invention, but do not exemplify the full scope of the invention.

### EXAMPLE 1

This example illustrates that by adding functionalities that enhance MS signals, such as arginine (R), at or near the C-terminus of the peptide chain, there may be significant enhancement of signal-to-noise ratios in MS and MS/MS acquisition. It is noteworthy that R is not included in the variable region of the peptide library. Therefore, key peptide fragment peaks can be identified by simply tracking the Y ions, for de novo peptide sequencing because all Y ions have one positively charged element. If the MS signal enhancer used is R, the loss of ammonia from guanidine group under MS/MS environment results in the generation of peak pairs with a mass difference of 17 amu. Two repetitive γ-amino butyric acid (GABA) groups were inserted between R and the variable region, so that R as a mass tag may reside distant from the binding site, which is desirable for ligand-related applications.

Another common problem is the presence of high intensity MALDI matrix clusters peaks of alpha-cyano-4-hydroxycinnamic acid (CHCA) in the range between 500 and 900 amu. This results in difficult identification of short peptide (4 to 6 amino acids) parent mass in the MS spectra. Addition of ammonium phosphate or nitrilotriacetic acid into sample solutions during the sample preparations can suppress matrix clusters of CHCA and improve signal-to-noise ratios in general. Another finding is that the parent mass of the released peptide can be increased to exceed that of the matrix clusters by locating a certain number of spacers such as y-amino butyric acid (GABA) group at the C-terminus of the peptide chain.

This example also demonstrates a new OBOC peptide library that has been successfully applied to screening the high-affinity capture agents for C-reactive protein (CRP).

General. N-methylpyrrolidone (NMP), diethylether, dichloromethane (DCM), and fmoc-γ-Abu-OH (GABA) were purchased from Merck. Non-natural Fmoc-protected amino acids (Fmoc-AAs) were purchased from GL Biochem Ltd (Shanghai. China). TentaGel S amino resin was purchased from Rapp Polymere. Alpha-cyano-4-hydroxycinnamic acid (CHCA) was purchased from Bruker. Unless otherwise specified, chemicals were purchased from Aldrich. MALDI-MS and MS/MS spectra were obtained using UltrafleXtreme TOF/TOF (Bruker). The microwave-assisted CNBr cleavage reaction was performed by a household microwave oven (model R-248J, 800 W, 2450 MHz) from Sharp, Inc.

Construction of peptide libraries. Random OBOC peptide libraries were synthesized using an automatic synthesizer, Titan 357 (AAPPTEC), via standard split-and-mix methods on polyethylene glycol-grafted polystyrene particles (TentaGel S—NH₂, 90 micrometers, 0.29 mmol/g, 2.86×10⁶ particles/g). Unless otherwise specified, non-natural D-stereoisomers were used at every possible position in the peptide sequence. For all the coupling steps, a standard solid-phase peptide synthesis method with Fmoc chemistry was used. The resin was swelled in NMP for 2 h in the collective vessel (CV). The coupling of Fmoc-methionine was initiated by addition of 2 equivalents of TBTU and 5 equivalents of DIEA. The coupling reaction was run for 30 min. Another 2 equivalents of Fmoc-methionine, 2 equivalent of TBTU, and 5 equivalents of DIEA were added and allowed to react for 30 min (“double coupling”). Following the coupling step, the particles were thoroughly washed (4×NMP) and treated with 20% piperidine in NMP (5 min and then 15 min with a fresh aliquot of deprotection solution). The resin was thoroughly washed (4×NMP, 4×DCM) and divided into multiple equal-mass aliquots for the next cycle of coupling in the reaction vessel (RV). With the coupling and Fmoc deprotection completed, the resins were combined in the CV. The procedures were repeated until the desired length of peptide was attained. The amino acid side chain protective groups were then removed by incubation in trifluoroacetic acid (95%), water (2.5%), and triisopropylsilane (2.5%) for 2 h. The library resin was then washed thoroughly with 5×DCM, 5×methanol, 5×deionized water, 5×methanol, 5×DCM, and then 5×diethyl ether. The resulting resin was dried under vacuum and stored at 4° C.

CNBr cleavage of peptides from single particles. A single particle was transferred to a microsized vial containing 10 microliters of deionized water. The reaction vessel was purged by argon for 15 min and then CNBr (10 microliters, 0.50 M in 0.2 N HCl solution) was added into the vessel. After additional purging by argon for 15 min, the vial was placed under microwave for 1 min. The resulting solution was concentrated under centrifugal vacuum for 10 min at 45° C. and then for 50 min at 60° C.

MALDI-MS and MS/MS analysis of peptides cleaved from single particles. To each vial or well were added CHCA (7 microliters, 0.4% solution in acetonitrile/water (70:30)) and then acetonitrile/water (7 microliters, 70:30 containing 0.1% trifluoroacetic acid (v/v) and 1 mM ammonium phosphate monobasic). A 2.5 microliter volume of the mixture solution was taken up to be spotted onto a 384-well MALDI plate, which was allowed to stand for 15 min to dry naturally. MALDI-MS and MS/MS were conducted with ultrafleXtreme™ MALDI-TOF/TOF mass spectrometer from Bruker Daltonics.

Library screening. For screening, the CRP protein was first labeled using the Alexa Fluor 647 protein labeling kit (A20173, Invitrogen) according to the supplier's protocol. First, a 2 mg/mL solution of CRP was dissolved in 0.1 M sodium bicarbonate (pH ˜8.3). Then 0.5 mL of this CRP solution was transferred into the vial of the reactive dye. The vial was capped and inverted a few times to fully dissolved the dye. The reaction mixture was stirred for 1 h at room temperature under dark conditions. The Alexa Fluor 647-labeled CRP was purified from the mixture using the size exclusion purification resin in the labeling kit. Purified and labeled CRP was characterized by UV-vis spectroscopy and SDS-PAGE. For the screen, 100 mg of library resin was transferred into an 8 mL Alltech vessel and preincubated in a blocking solution, 0.05% NaN₃, 0.1% Tween 20, and 0.1% BSA in PBS buffer (pH 7.4), for 1 h on a 360° shaker at 25° C. The buffer solution was drained by vacuum. and then 5 mL of 10 nM dye-labeled CRP diluted in blocking solution was added to the swollen resin. The resulting mixture was incubated for 15-18 h on a 360° shaker at 25° C. The liquid was drained by vacuum, and nonspecifically bound proteins were eliminated by washing three times with blocking solution and three times with 0.1% Tween 20 in PBS buffer sequentially. Last, the resin was washed six times with PBS buffer. After stringent washing, 200 mg of the assayed library resin was transferred into a sample vessel of COPAS Plus (Union Biometrica) and diluted with 200 mL of PBS buffer (pH 7.4). Two-step sorting was applied. In the second sorting, positive particles were directly sorted into a 96 titer well plate with cone-shaped wells. CNBr cleavage and MALDI-MS and MS/MS followed.

Synthesis of peptides with acetylene modification at C-terminus in bulk. Bulk synthesis of hit peptides was performed on Fmoc-Rink amide MBHA resins (0.58 mmol/g) on a typical resin scale of 0.2 g per sequence. With the desired sequence of peptide attained, the resin was treated in trifluoroacetic acid (95%), water (2.5%), and triisopropylsilane (2.5%) for 2 h. The cleavage cocktail was concentrated in a continuous flow of nitrogen, and the crude peptides were precipitated in diethyl ether. The resulting white solid then purified to >95% by HPLC on a C₁₈ reversed-phase preparative column. The pure peptides were used for affinity measurements and also for click reaction with the biotin group for dot blot experiments.

Bulk synthesis of biotin with azide modification at C-terminus. Bulk synthesis of biotin was performed on Fmoc-Rink amide MBHA resins (0.58 mmol/g) on a typical resin scale of 0.2 g per sequence. FMOC-Lys(Mtt)-OH was first coupled to Rink amide resin. Then, selective deprotection of Mtt group was performed with deprotection of Mtt by reaction of particles with TFA/TIS/DCM (1/5/94 ratio) for 2 hours. For the next coupling step, Biotin-NHS with DIEA was added and the mixture was stirred for 2 hours. After coupling, the resin was treated in trifluoroacetic acid (95%), water (2.5%), and triisopropylsilane (2.5%) for 2 h. The cleavage cocktail was concentrated in a continuous flow of nitrogen, and the crude peptides were precipitated in diethyl ether. The resulting white solid then purified to >95% by HPLC on a C₁₈ reversed-phase preparative column.

Synthesis of biotinylated peptides in bulk by click reaction. 1 micromolar solution of the biotinylated peptides and the peptide ligand (each produced as discussed above) were prepared in 500 microliters of tert-butanol:water (1:1) mixture in a 1.5 mL microcentrifuge tube. 10 microliters of 0.5 M copper sulfate solution and 12 microliters of 0.5 M sodium ascorbate solution was then added into the reaction vessel in the presence of argon and allowed to shake on a 180° shaker overnight. After reaction, all solvents in the reaction vessel were evaporated using a centrifugal concentrator GeneVac EZ2 Plus and dissolved in acetonitrile/water mixture for purification on a C₁₈ reversed phase preparative column.

Affinity measurements. Affinity measurements were performed using a Biacore T100 system and research grade CM5 sensor chips (GE Heathcare). The instrument was primed with HBS-EP+ (GE Heathcare) buffer. Flow cell 1 (or 3) was used as a reference to subtract nonspecific binding, drift, and the bulk refractive index, while flow cell 2 (or 4) was immobilized with CRP following standard procedures. A 1:1 mixture of 0.4 M EDC and 0.1 M NHS was used to activate flow cell 2 (or 4), and 0.1 mg/mL CRP solution was injected. Blocking of the remaining activated groups was done with a 1 M solution of ethanolamine (pH 8.5). CRP was immobilized onto the sensor chip surface by approximately 5000 response units (RU). The instrument was then primed using running buffer (HBS-EP+). Each of the 6-mer ligand candidates identified were dissolved in HBS-EP+ buffer to produce 5 micromolar peptide stock solutions for each peptide, which were serially diluted by a factor of 2 to produce a concentration series down to 2 nM. For a given affinity measurement, these series of peptide solutions successively were injected into flow cell 2 (or 4) for 3 min of contact time, 5 min of dissociation time, and 3.5 min of stabilization time using a flow rate of 100 microliter/min at 25° C. Flow cell 2 (or 4) was regenerated by glycine 2.5 (GE Healthcare) after injection of each peptide solution.

Dot blot selectivity/sensitivity assays in serum. The affinity of the biligand capture agents for CRP was demonstrated through the use of dot blot experiments in 5% nonfat dry milk in TBS-T (25 mM Tris, 150 mM NaCl, 2 mM KCl, 0.5% Tween 20 (pH 8.0)). CRP solution was prepared as 10 mg/mL stocks in PBS buffer (pH 7.4). A dilution series of CRP solution was applied to a nitrocellulose membrane, typically ranging from 20 micrograms to 2 ng per spot. The membrane was blocked at room temperature for 2 h in 5% nonfat milk/TBS-T. The membrane was then washed with TBS-T. The biotinylated 6-mer solution was prepared at 1 micromolar in 5% nonfat milk/TBS-T and incubated over the membrane for 2 h at room temperature. After washing three times with TBS-T for 10 min, 1:3000 streptavidin-HRP (Abeam) prepared in 0.5% milk/TBS-T was added to the membrane and incubated for 2 h. After washing three times with TBS-T for 10 min, the membrane was treated with chemiluminescent reagents (Amersham ECL plus Western blotting detection reagents, GE Healthcare) and then immediately developed on film.

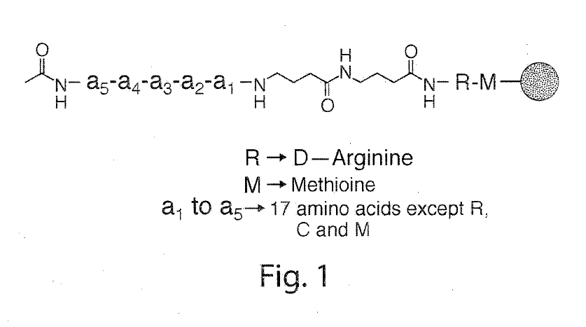

Structure of OBOC library used for validation. The library structure used in this example was designed for efficient screening of particle-based peptide libraries by solving these problems: weak ionization of certain types of peptides, overlapping of parent masses with matrix clusters, and occurrence of hits due to non-specific interactions. A peptide chain with the structure shown in FIG. 1 was synthesized and tested.

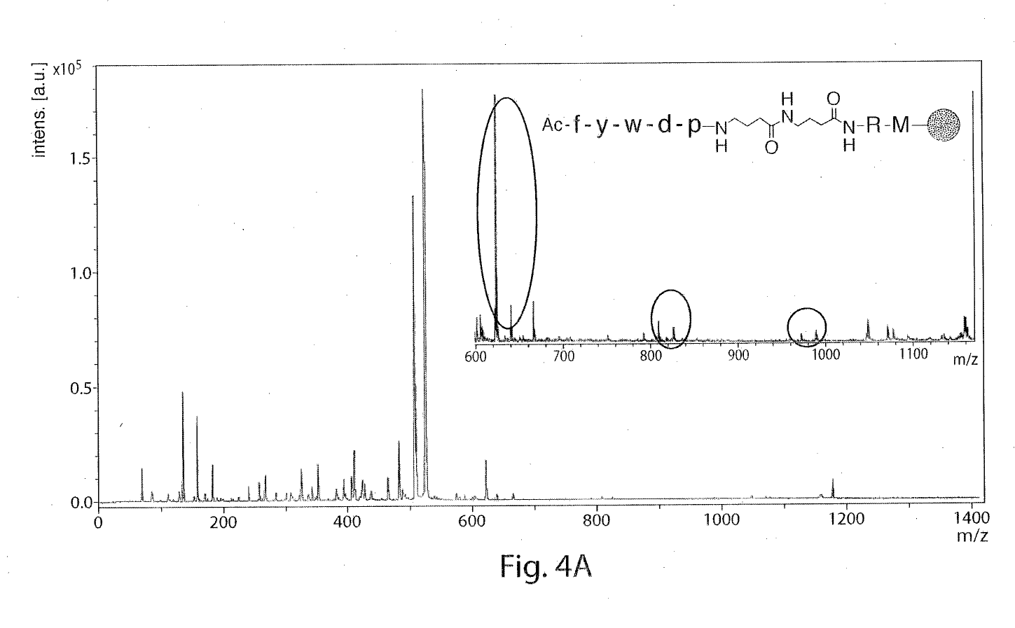

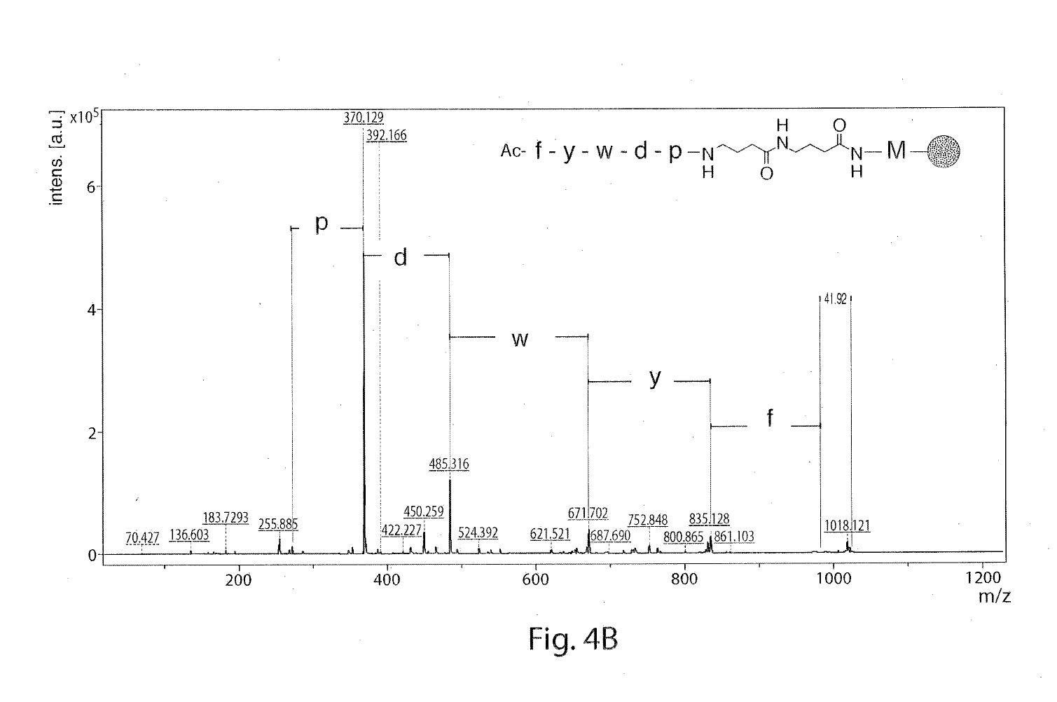

In this structure, an acetyl group (Ac) was introduced at N-terminus of the peptide chain in the structure. To avoid overlapping of parent masses with matrix cluster peaks, 2 repetitive units of a gamma-amino butyric acid (GABA) spacer were inserted to increase the parent mass. In addition, there was incorporation of R as a MS signal enhancer next to the CNBr-cleavable methionine. The positively charged guanidine of R may enhance ionization efficiency during MS and MS/MS measurements, which should increase signal intensities in general. The magnitude of increase was dependent on the sequence of peptide involved. For example, in a sequence FYWDP (SEQ ID NO: 1) shown in FIGS. 4A and 4B, the signal enhancement provided by R was almost 10-fold. R was not included in diversity elements of the peptide library to reduce non-specific interactions between peptides and the target protein during the screening. With this structure in OBOC library, all fragmented Y ions in MS/MS spectra contained just one R between GABA and homoserine lactone. This resulted in the formation of pairs of peaks for key Y ions due to the loss of ammonia (-17 amu) from guanidine group of R during the fragmentation for MS/MS experiments. Consequently, the hit peptides could be sequenced with enhanced accuracy and ease.

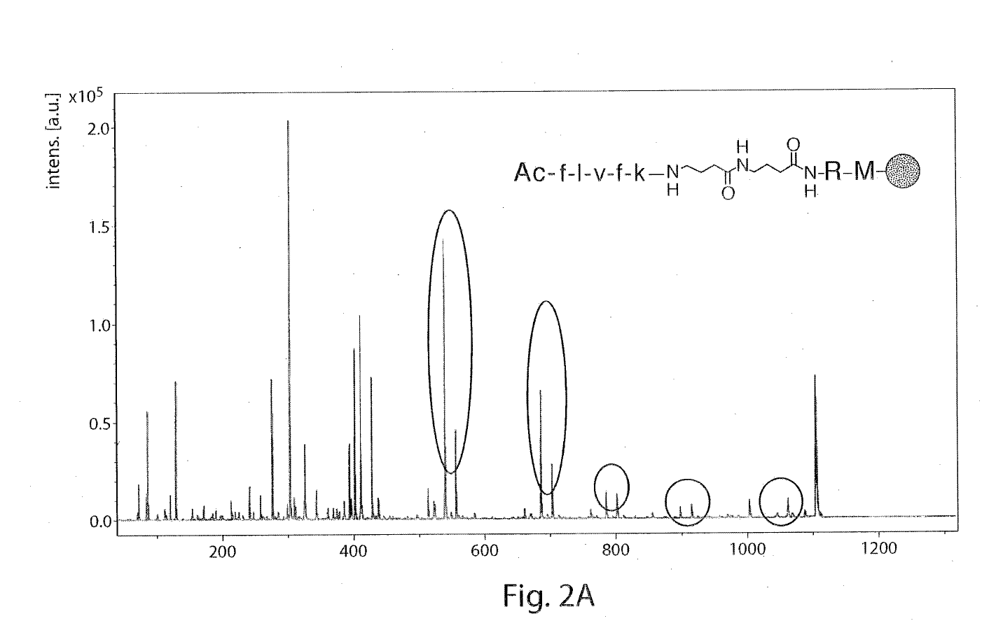

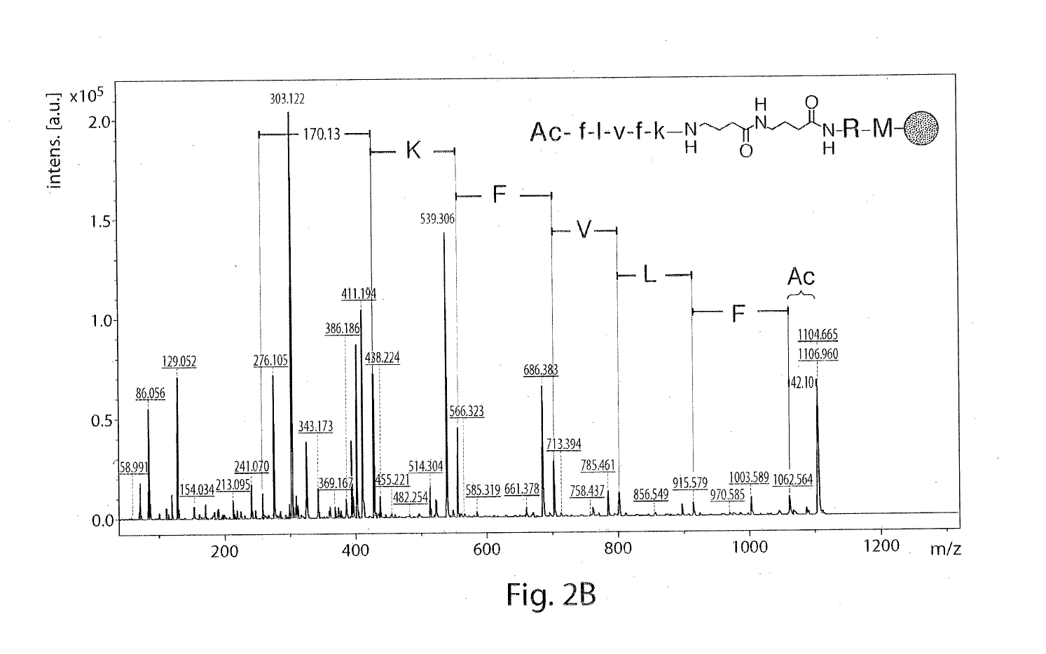

Evaluation of the designed OBOC library for de novo peptide sequencing. FIG. 2A shows a typical MS/MS spectrum obtained from a designed particle. It was straightforward to identify the key Y ions as well as the unwanted peaks around the parent peak with reasonably high signal intensity. All Y ions in the MS/MS spectra should contain just one R between GABA and homoserine lactone that enhances the ionization of all fragments. In addition, the Y ions could be identified due to the loss of ammonia (−17 amu) from R, which resulted in pairs of peaks occurring for key mass values. Sequencing could be performed after recognizing peak pairs of Y ions by taking the mass difference between the peaks as illustrated in FIG. 2B.

FIG. 2A shows an MS/MS spectrum of a peptide FLVFK (SEQ ID NO: 2) showing the peak pairs of the key peptide fragments. FIG. 2B shows an MS/MS spectrum of a peptide FLVFK (SEQ ID NO: 2) showing that sequencing can be performed by taking the mass difference between fragments.

The structural advantages of this spacer also ensures that the particle-based library can be applied to specific ligands or capture agents. The linear spacer of gamma-amino butyric acid (GABA) had minimal interaction with protein binding sites in general. Furthermore, the modifications such as the incorporation of R are all carried out between GABA and methionine at positions near the C-terminus to minimize modification-protein interaction.

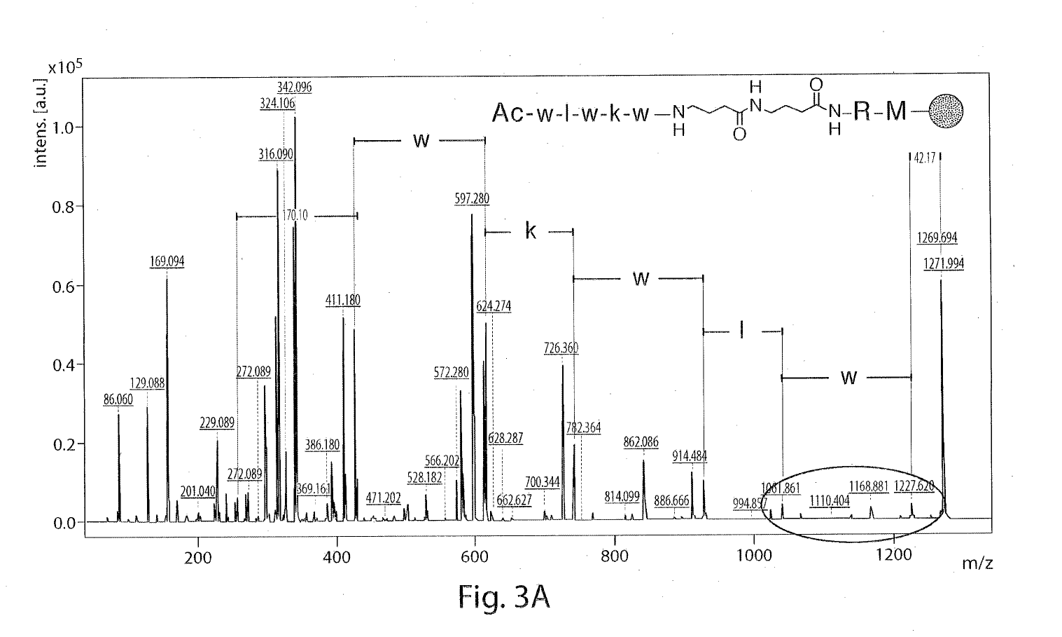

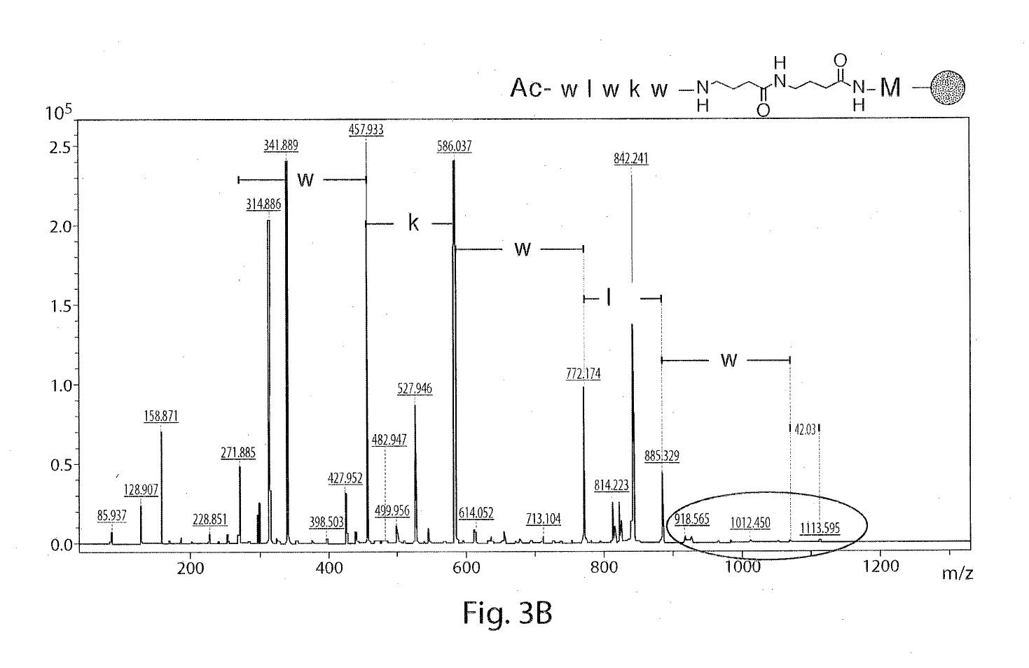

To further show that placing R next to methionine may be important in designing a library structure, known sequences were synthesized with R between GABA and methionine. In general, the quality of the MS and MS/MS spectra obtained was not good with the same MALDI-MS conditions. For the sequence WLWKW (SEQ ID NO: 3), the peak intensities near the parent mass were too low, which made the sequencing more challenging. This finding can be seen when FIGS. 3A and 3B are compared. The key Y ion peaks in FIG. 3A were higher in intensity and occurred in pairs for easy recognition. Generally, peptides could be more easily sequenced with the newly designed library. This observation is also evident by comparing FIGS. 4A and 4B, which shows MS/MS spectra of FYWDP (SEQ ID NO: 4) synthesized with two different types of structure.

FIG. 3A shows an MS/MS spectrum of a peptide WLWKW (SEQ ID NO: 3) with high intensity peaks for all key mass values, with clear peaks near the parent mass as highlighted in the circle. FIG. 3B shows an MS/MS spectrum of a peptide WLWKW (SEQ ID NO: 3) with no R in the chain. Peaks near parent mass showed very low intensities as highlighted in the circle. FIG. 4A shows an MS/MS spectrum (2000 laser shots) of a peptide FYWDP (SEQ ID NO: 4) with R in the chain, with peaks near parent mass showing reasonable intensity. FIG. 4B shows an MS/MS spectrum (10,000 laser shots) of a peptide FYWDP (SEQ ID NO: 4) with no R in the chain with most of the peptide fragments showing significantly low intensities.

This structure also worked well for the peptides containing negatively charged amino acids, such as glutamic acid (E) and aspartic acid (D), in their sequence. Due to the negative charge, ionization is often inefficient and intensities of key peaks for sequencing are often very low. With the signal enhancer R, the spectra for these peptides were clear as well. An example of the MS/MS spectrum obtained from such a peptide is shown in FIG. 4A. FIG. 4B shows the peptide with no R in the chain. The spectrum was almost flat for most key mass values. FIG. 4A was obtained with 2000 laser shots while FIG. 4B was obtained with 10,000 laser shots on MALDI-MS experiments. Without R, ionization was extremely weak, resulting in low intensity of peaks. Signal-to-noise ratio can increase by 5- to 10-folds with the incorporation of R for these acidic peptides.

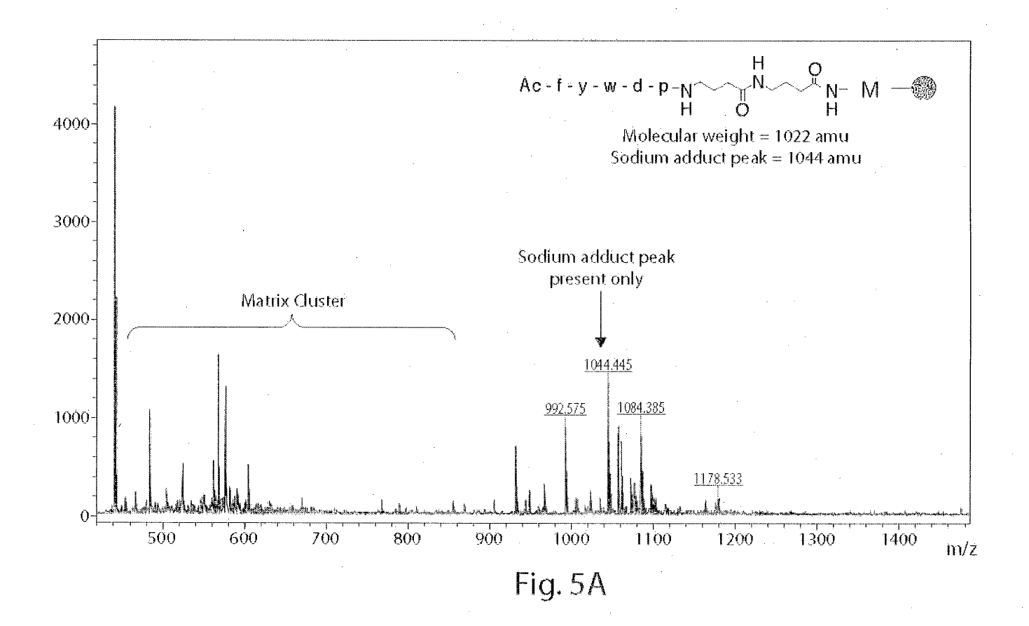

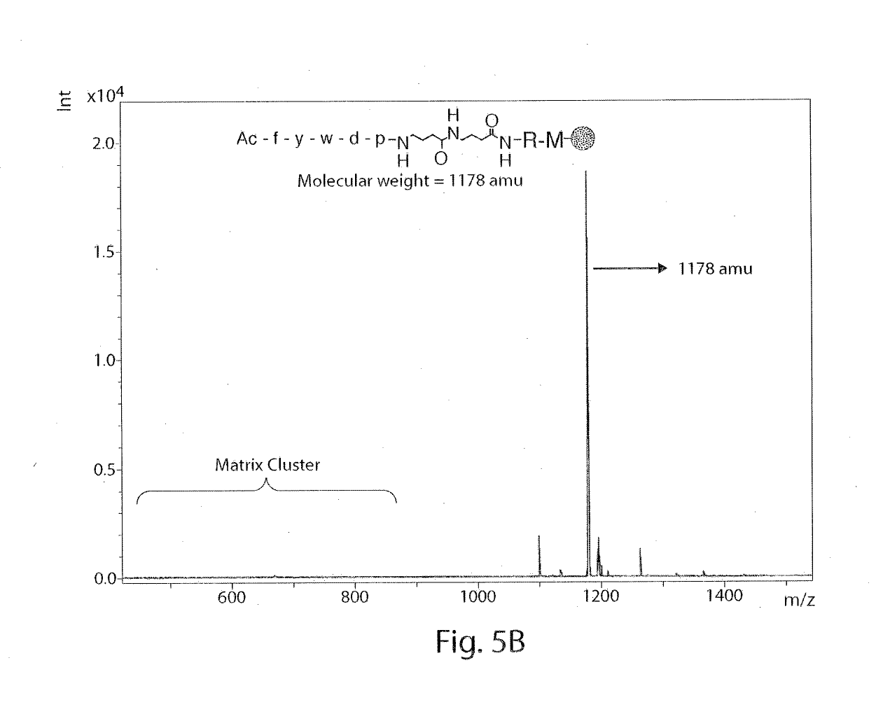

The addition of GABA spacers shifted the parent mass to a range which did not overlap with the CHCA matrix cluster peaks. Moreover, 1 mM solution of ammonium phosphate added during the sample preparation suppressed the matrix cluster peaks. With these factors combined, clear MS spectrum could be obtained to identify the parent mass. This may be seen by comparing the spectra. For instance, FIG. 5A shows a MS spectrum of a peptide FYWDP (SEQ ID NO: 1) without R between GABA and methionine The matrix clusters appeared clear due to the low intensity of parent mass of peptide. While the actual mass of 1022 amu was not observed, only the sodium adduct peak of 1044 amu was observed. The actual peak was not observed possibly due to weak ionization, instead favoring formation of the sodium adduct as it is a negatively charged peptide.

FIG. 5B shows that with R present between GABA and methionine, the spectrum was clearer, with the same sequence showing only one prominent peak at 1178 amu. The intensity of the parent peak in FIG. 5B was considerably high even though it came out of an acidic peptide. Hence, with the positively charged R inserted, the intensity of parent masses maybe greatly enhanced.

Thus, based on the results obtained above, the newly designed OBOC library worked well for efficient de novo sequencing. The quality of spectra was sufficient to identify most types of peptides, even including acidic peptides which are typically more problematic.

Application of the novel OBOC library to real screening. Using the novel library, screening was carried out for C-reactive protein (CRP) labeled with Alexa Fluor 647 fluorescence dye. The particle-based peptides interacted at equilibrium with a target protein to give a reasonable fluorescence level during the automated sorting of hit particles by running COPAS Plus. The aim of the screening exercise was to identify 6-mer ligands via two generations of screens. Firstly, CRP was screened employing the novel 5-mer comprehensive library, of which details are shown in FIG. 1. Issues occurring in the screening were also taken into consideration for the design of the 5-mer comprehensive library shown in FIG. 1. To reduce the size of the comprehensive library, a 5-mer library was used instead of the corresponding 6-mer. A library of about 800 mg in size was synthesized with one copy per each sequence. About half of the library was used for the initial screen. The screening of a portion of a whole library produced more reliable and comprehensive screening results. In each of the amino acid positions. R was not included to reduce nonspecific interactions. To avoid overlapping of parent masses with matrix peaks, 2 units of GABA spacer were added to increase the parent mass. Desirably, this moiety had minimal interaction with the target protein.

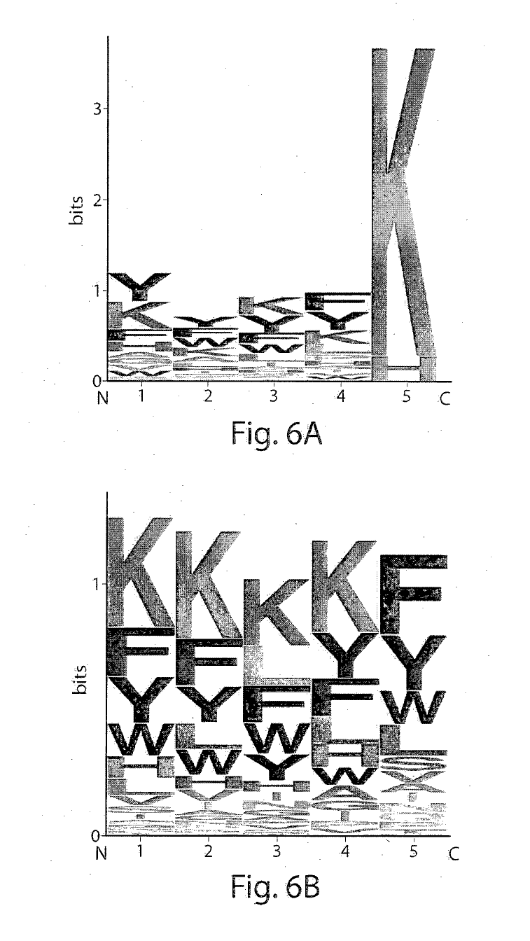

The peptides of hit particles from the comprehensive 5-mer library were analyzed using a semi-automatic sequencing method. In the initial screening of the comprehensive 5-mer peptide library, the sequencing results showed a good homology with repeating motif sequences, such as KF. KY, KW, and KFY. To further analyze the results, the hit sequences were divided into two groups based upon the nature of amino acids at N-terminus, in terms of being hydrophilic or hydrophobic, respectively. The relative dominance of amino acids in each position is highlighted in the font histograms shown in FIG. 6A (hydrophilic amino acids at the N-terminus) and FIG. 6B (hydrophobic amino acids at the N-terminus).

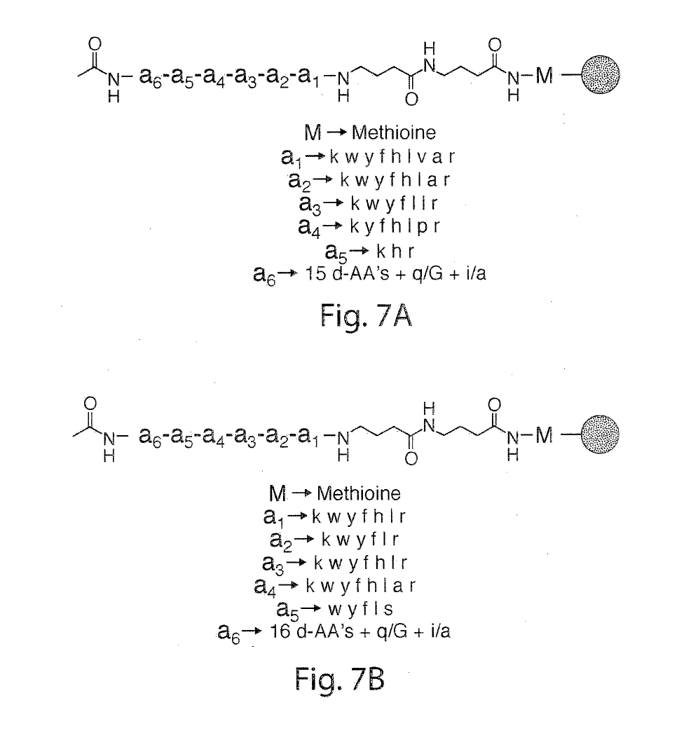

Based on the occurrence of amino acids in screening the initial general library, two 5-mer focused libraries were proposed from the two groups. For enhanced binding affinity, the 5-mer focused library was elongated by adding one more amino acid as the sixth diversity element at the N-terminus of the both libraries. This new position had a diversity of 18 d-amino acids. Then, the two focused 6-mer OBOC peptide library, as illustrated in FIGS. 7A and 7B, were proposed from the positive 5-mer peptides from the first-generation screen. In the focused libraries, R was included as a diversity element for all positions containing K as one of dominant amino acids.

FIG. 7A shows the structure of the 6-mer focused library A, used in screening, with hydrophilic amino acids at a₅ of the peptide. FIG. 7B shows the structure of the 6 -mer focused library B, used in screening, with hydrophobic amino acids at a₅ of the peptide

Next, screening for CRP was conducted with using the entire synthesized 6-mer focused library. Table 1 shows the conditions used for screening. Sequences occurring more than once are selected and synthesized for validation by surface plasmon resonance (SPR) and dot blot experiments. Table 2 shows the sequences selected via the focused library screening.

TABLE 1
Conditions used for screening
Screening Conditions

| | | Number | | |
|---|---|---|---|---|
| Type of | | of runs * | | |
| Library | Concentration | Mass of | Size of | |
| used | of CRP | particles used | Library | Temperature |
| 5-mer | 10 nM | 2 * 100 mg | 800 mg | 25° C. |
| Comprehensive | | 1 * 200 mg | | |
| 6-mer Focused | 10 nM | 2 * 200 mg | 400 mg | 25° C. |
| Library A | | | | |
| 6-mer Focused | 10 nM | 2 * 200 mg | 400 mg | 25° C. |
| Library B | | | | |

TABLE 2
8 CRP 1° ligand candidates proposed from 6-mer focused library A and B
screening

| | | | | | | |
|---|---|---|---|---|---|---|
| Candidate | a₆ | a₅ | a₄ | a₃ | a₂ | a₁ |

Focused Library A

| | | | | | | |
|---|---|---|---|---|---|---|
| 1 | W | K | F | R | Y | K |
| 2 | G | R | F | R | L | F |
| 3 | L | R | L | K | Y | R |
| 4 | Y | R | R | W | F | R |

Focused Library B

| | | | | | | |
|---|---|---|---|---|---|---|
| 1 | L | Y | R | R | F | Y |
| 2 | L | Y | F | R | R | W |
| 3 | K | F | R | F | R | Y |
| 4 | L | Y | F | Y | K | R |

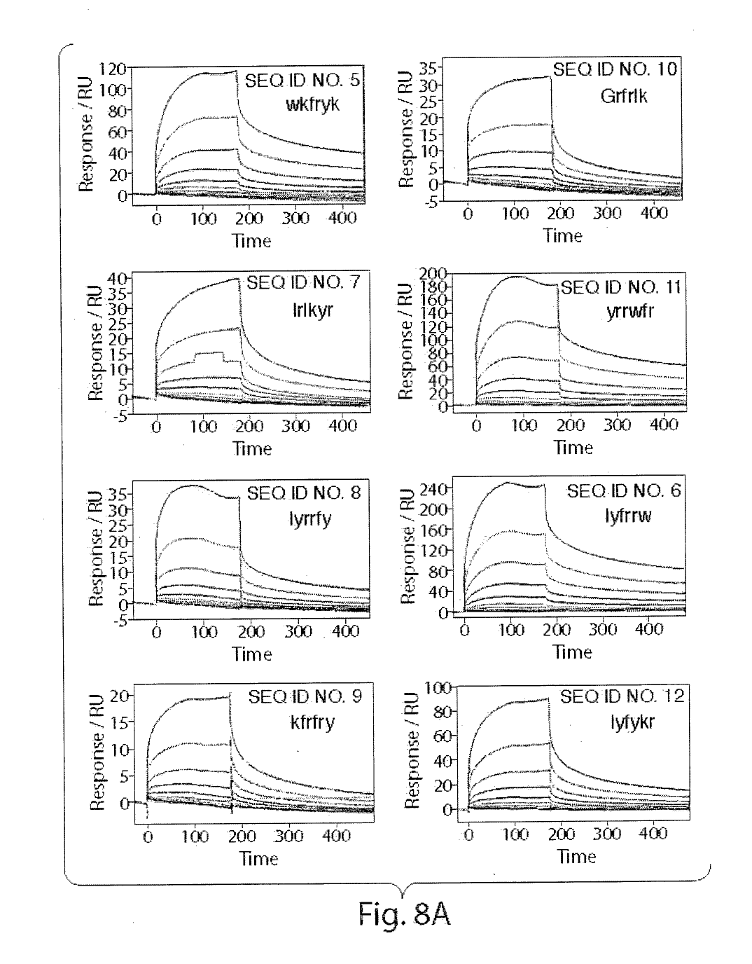

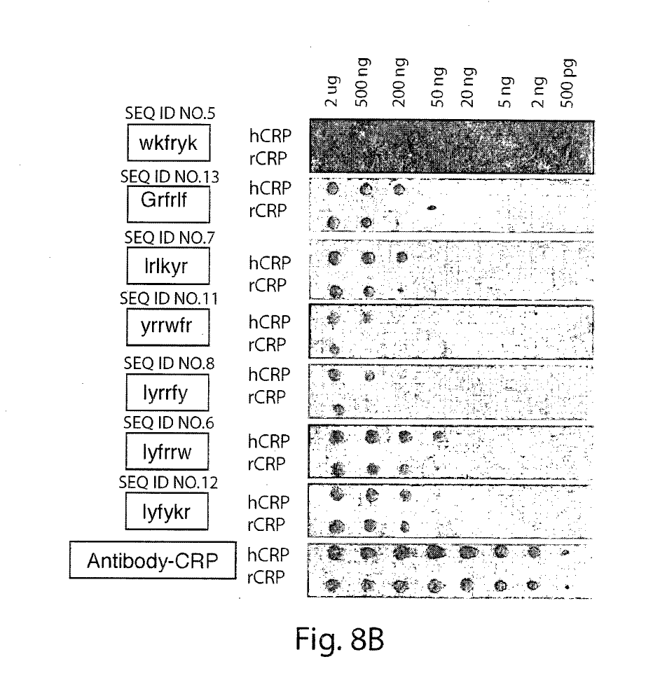

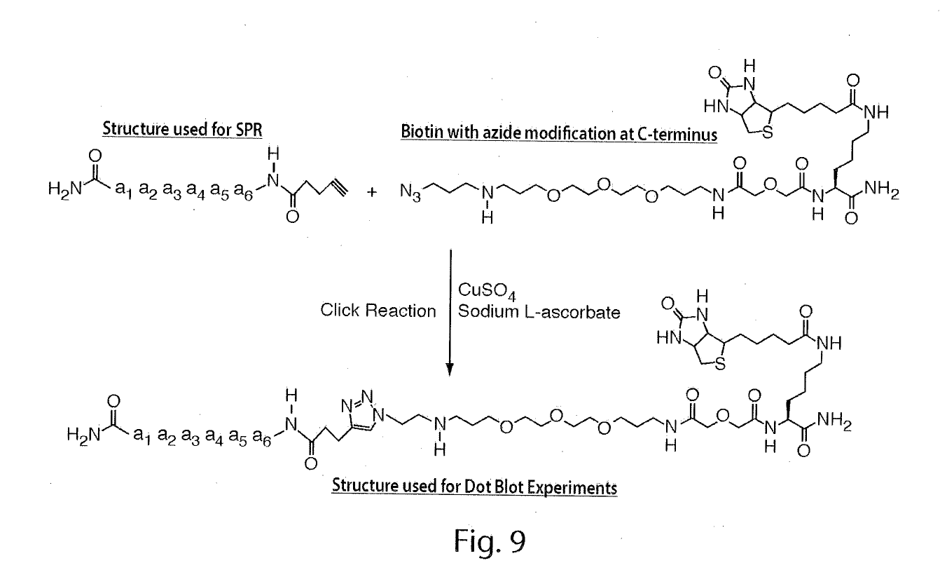

SPR and dot blot studies were performed to validate the sequences obtained from screening focused libraries A and B. FIG. 9 shows the reaction scheme to synthesize the structures for dot blot validation from the peptide structure used in SPR measurements. FIGS. 8A and 8B show the results from the two validation studies, respectively. FIG. 8A shows SPR results of the 8 peptide candidates. FIG. 8B is a dot bolt of the results of the 7 peptide candidates.

From the SPR and dot blot results, all peptide candidates derived showed good binding affinity to CRP. Among them, the peptides WKFRYK (SEQ ID NO: 5) and LYFRRW (SEQ ID NO: 6) showed the highest response both in dot blot study and SPR. Therefore, the new screening strategy with the library structure in this example proved to be successfully employed for actual screening against target proteins.

In this example, an OBOC peptide library was designed for more efficient screening of OBOC peptide libraries. This newly-designed OBOC library also aimed to solve several problems, such as non-specific interactions, weak ionization of certain peptides and peptide fragments, and overlap of parent masses with matrix clusters. To minimize non-specific interactions, R was excluded for the construction of peptide libraries. To enhance ionization of peptides and peptide fragments, one R was incorporated at between GABA and methionine, so that all the Y ions have one R for enhanced ionization. Two GABA groups were used as a spacer between R and diversity region to minimize the interference of R in screening and to avoid the overlap of parent masses with matrix clusters by increasing the mass of parent peptide.

In order to evaluate the novel OBOC peptide libraries for efficient de novo sequencing, several known peptides were synthesized on single particles and investigated. All peptides released from the newly designed peptides on particles resulted in enhanced peptide sequencing with enhanced ionization, less overlap with matrix clusters and easier identification of parent masses and peptide fragments.

When the OBOC 5-mer peptide library was applied to an actual screening against CRP, the results showed excellent homology in peptide sequence. To generate the focused libraries, a new approach was used that incorporated R at all positions where K appeared as one of dominant amino acids, and to increase peptide size from 5-mer to 6-mer by adding 18 amino acids at N-terminus. All the hit peptides obtained from the screening of the focused libraries showed good responses to the target CRP, both in SPR and dot blot experiments.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one. A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving.” “holding.” “composed of” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a particle having a peptide attached thereto, having a structure:
(particle)-M-Q-Zₙ—X-J,
wherein:
M is a methionine residue;
Q is a group able to enhance intensity and/or sensitivity of mass spectrometry;
X comprises a plurality of amino acid residues;
n is a positive integer;
J is an endgroup; and
Z is a spacer having a structure: 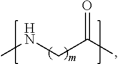
wherein m is a positive integer greater than or equal to 3.

2. The article of claim 1, wherein X comprises between 4 and 10 amino acid residues, inclusively.

3. The article of claim 1, wherein X comprises 5 amino acid residues.

4. The article of claim 1, wherein the particles have an average diameter of less than about 1 mm.

5. The article of claim 1, wherein Q is positively charged and/or is an arginine residue.

6. (canceled)

7. The article of claim 1, wherein Q comprises Br and/or a —NH₂ moiety.

8. (canceled)

9. The article of claim 1, wherein J is acetyl.

10. An article, comprising:
a collection of particles having peptides attached thereto, at least some of the particles consisting essentially of one type of peptide, the collection including at least 20 distinguishable types of peptides, wherein the particles and the peptides have a structure:
(particle)-M-Q-Zₙ—X¹—X²—X³—X⁴—X⁵-J,
wherein:
M is a methionine residue;
Q is a group able to enhance intensity and/or sensitivity of mass spectrometry;
Zₙ is either a covalent bond or a spacer that does not contain a naturally-occurring amino acid residue, wherein n is a positive integer greater than or equal to 2 when Z is a spacer;
X¹, X², X³, —X⁴ and X⁵ are each independently amino acid residues excluding arginine; and
J is an endgroup.

11. The article of claim 10, wherein J is acetyl.

12. The article of claim 10 or 11, wherein Zₙ is linked to each of R and X¹ via amide linkages.

13. The article of claim 10, wherein Z comprises an aromatic group and/or has a structure: 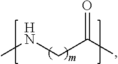
wherein m is a positive integer.

14. (canceled)

15. The article of claim 10, wherein Q is an arginine residue.

16. An article, comprising:
a particle having a peptide attached thereto, having a structure:
(particle)-M-R—Z₂—X¹—X²—X³—X⁴—X⁵-J,
wherein:
M is a methionine residue;
R is an arginine residue;
X¹, X², X³, X⁴, and X⁵ are each independently amino acid residues excluding arginine;
J is an endgroup; and
Z has a structure:

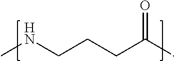

17. An article, comprising:
a particle having a peptide attached thereto, having a structure:
(particle)-M-X¹—X²—X³—X⁴—X⁵—Zₙ-Q-J,
wherein:
M is a methionine residue;
X¹, X², X³, X⁴, and X⁵ are each independently amino acid residues excluding arginine and cysteine;
Zₙ is either a covalent bond or a spacer that does not contain a naturally occurring amino acid residue, wherein n is a positive integer when Z is a spacer;
Q is either a covalent bond between Zₙ and J, or a group able to enhance intensity and/or sensitivity of mass spectrometry; and
J is an endgroup.

18. The article of claim 17, wherein X¹, X², X³, X⁴, and X⁵ are each independently amino acid residues excluding arginine, cysteine, and methionine.

19. The article of claim 17, wherein Z has a structure: 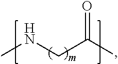
wherein m is a positive integer.

20. The article of claim 17, wherein n is 1 or 2.

21. (canceled)

22. The article of claim 17, wherein m is 3.

23. The article of claim 17, wherein Z comprises an aromatic group.

24. The article of claim 17, wherein Q is an arginine residue.

25. An article, comprising:
a particle having a peptide attached thereto, having a structure:
(particle)-M¹-X¹—X²—X³—X⁴—X⁵—Zₙ-Q-J,
wherein:
M¹ is a cleavable linker residue;
X¹, X², X³, X⁴, and X⁵ are each independently amino acid residues excluding arginine and cysteine;
Zₙ is either a covalent bond or a spacer that does not contain a naturally occurring amino acid residue, wherein n is a positive integer when Z is a spacer;
Q is either a covalent bond between Zₙ and J, or a group able to enhance intensity and/or sensitivity of mass spectrometry; and
J is an endgroup.

26. The article of claim 25, wherein M¹ is methionine, tryptophan, aspartic acid-proline, asparagine-glycine, or cysteine.

27-30. (canceled)

31. The article of claim 25, wherein M¹ is cleavable upon exposure to a cleaving agent.

32. The article of claim 31, wherein the cleaving agent comprises cyanogen bromide, BNPS-skatole, formic acid, hydroxylamine, and/or 2-nitro-5-thiocyanobenzoic acid.

33-36. (canceled)

37. The article of claim 25, wherein Z comprises an aromatic group.

38. The article of claim 25, wherein Q comprises an arginine residue, a lysine residue, Br, and/or Cl.

39-41. (canceled)

42. A method, comprising exposing the article of claim 1 to a target molecule.

43. The method of claim 42, wherein the target molecule is a protein.
